(19) United States
(12) Patent Application Publication
(10) Pub. No.: US 2013/0160763 A1
(43) Pub. Date: Jun. 27, 2013

(54) METHOD OF RELIEVING PAIN ASSOCIATED WITH FRACTURED RIBS

(71) Applicant: William T. Wyatt, Reform, AL (US)

(72) Inventor: William T. Wyatt, Reform, AL (US)

(21) Appl. No.: 13/465,759

(22) Filed: May 7, 2012

Related U.S. Application Data
(60) Provisional application No. 61/580,483, filed on Dec. 27, 2011.

(51) Int. Cl. A61M 16/00

(52) U.S. Cl. 128/202.16

(57) ABSTRACT

A method of relieving pain resulting from breathing in a patient with fractured ribs, wherein the breathing causes movement of the fractured portion of the ribs. A positive airway pressure machine is attached to the nose, mouth, or both of the patient. The patient turns the machine on to produce a positive airway pressure in the airway or airways of the lungs and adjusts the positive airway pressure to a level where the positive airway pressure relieves pain produced by breathing. The method allows a patient with fractured ribs to sleep comfortably without the pain associated with the breathing-related movement of the fractured portion of the ribs, and also promotes the healing and recovery of the fractured ribs.

### CROSS-REFERENCE TO RELATED APPLICATIONS

This application claims priority of U.S. Provisional Application Ser. No. 61/580,483 filed Dec. 27, 2011, the disclosure of which is incorporated herein by reference.

### FIELD OF THE INVENTION

The present invention relates to the field of continuous positive airway pressure devices and methods and, more particularly, to a method of relieving pain associated with breathing in the presence of fractured ribs by using externally applied continuous positive airway pressure and devices related thereto.

### BACKGROUND OF THE INVENTION

Whenever ribs are fractured, either accidentally or through a medical procedure, there is little that can be done to prevent or relieve the pain associated with the recovery and healing of the ribs, or to promote the healing of the fractured ribs. Taping the thorax to stabilize the ribs is not recommended because it is associated with the occurrence of pneumonia. Patients receive pain medication and are instructed to sleep in a recliner and are advised that it will take three to six months for the pain to subside. A relatively serious problem is that the pain interferes with a patient's ability to get an adequate amount of sleep, which may interfere with the healing process. The pain is usually caused by the movement of the fractured portion of the ribs during breathing. What is needed is a method of relieving pain during breathing associated with movement of the fractured portion of the ribs, particularly during sleeping, and which promotes the healing and recovery of fractured ribs.

### SUMMARY OF THE INVENTION

The present invention provides a method of relieving pain produced by breathing in a patient with fractured ribs using a non-ventilating positive airway pressure machine. A patient attaches the positive airway pressure machine to his or her nose, mouth, or both and turns on the positive airway pressure to produce an expiratory positive airway pressure in his or her airway or airways of the lungs throughout the expiratory cycle. The patient adjusts the positive airway pressure produced by the positive airway pressure machine to a level where the positive airway pressure relieves pain produced by breathing in the patient. The patient can use the method until no movement of the fractured portion of the ribs is detectable by the patient during normal breathing in the absence of the positive airway pressure machine. In addition to relieving pain during breathing, the method accelerates the healing of the fractured ribs of the patient.

An advantage of the invention is a simple and safe method for relieving pain in a patient with fractured ribs that occurs during breathing and that is associated with movement of the fractured portion of the ribs.

Another advantage is a method which promotes the healing and recovery of fractured ribs.

Another advantage is a method which allows a patient with fractured ribs to sleep comfortably without the pain associated with breathing.

Another advantage is a method which helps prevent pneumonia in the patient by producing a positive pressure in the lungs and prevents the lungs from collapsing.

### DETAILED DESCRIPTION OF THE INVENTION

While the following description details the preferred embodiments of the present invention, it is to be understood that the invention is not limited in its application to the details of the method described herein, since the invention is capable of other embodiments and of being practiced in various ways.

The method of the present invention uses, preferably, continuous positive airway pressure (CPAP) or bi-level positive airway pressure (BPAP) devices or machines, or known variations thereof. These machines are designed to maintain a positive pressure in the airway of the lung to prevent sleep apnea. During sleep apnea the airway is prone to narrowing or collapsing. The positive air pressure keeps the airway open and prevents the occurrence of obstructive sleep apnea syndrome. However, U.S. Pat. No. 7,810,496 teaches that the surplus inspiratory positive airway pressure at the end of inspiration that occurs with a non-ventilating BPAP machine leads to uncomfortable and potentially harmful hyperinflation of patient's lungs. This patent further teaches that the administration of expiratory positive airway pressure throughout the expiratory cycle with the BPAP machine undesirably contributes to the breathing work that a patient must perform during exhalation. These effects produced by a non-ventilating BPAP machine suggest that this machine would not be useful for relieving the pain associated with breathing in a patient with broken ribs, and would probably exacerbate the pain and delay the process of healing of, and the recovery from, the broken ribs.

The method of the present invention uses positive airway pressure devices known in the art. The device may typically include a drive system for generating a pressurized air stream variably at a volume and pressure as desired, and may include a delivery system of fittings, tubing/hoses, and masks in order to deliver the pressurized air into the breathing system of a patient. The device may include various electrical and electronic control systems in order to turn the machine on and off, control the air pressure or motor speed, and the like. Other systems may be incorporated to accommodate the valving of air flows to and from the lungs of a patient. The power system for the device includes wall power, converted DC power from an AC wall outlet through a DC power supply, DC power from an automobile outlet through a DC power supply, a battery, or the like.

The method of the present invention comprises 1) providing a positive airway pressure machine; 2) attaching the machine to the mouth, or nose, or both of a patient having fractured ribs so that a positive pressure of air is applied to the airway and lungs of the patient; 3) increasing the positive airway pressure to a level where pain associated with breathing is decreased, maximally reduced, or not detectable and, if desired, where the movement of the fractured portion of the ribs is decreased, maximally reduced, or not detectable; and 4) using the device, preferably for at least two days, or until the patient does not detect the movement of the fractured portion of the ribs during normal breathing in the absence of the positive airway pressure machine.

The positive airway pressure device can produce continuous or intermittent air pressure in the airway(s) of the patient at levels from 4 to 30 cm H₂O. The patient applies the device to his or her airway and adjusts the air pressure produced by the device to a level where the pain associated with breathing is ameliorated as desired. Anyone can assist the patient, if desired, in adjusting the positive air pressure. However, the patient can, preferably, adjust the positive airway pressure by himself or herself. The positive airway pressure device can be constructed as a small, portable device that can be powered by any power source known in the art.

### EXAMPLE

A patient had fallen and complained that his ribs may have been broken. X-ray examination revealed that the number nine and ten ribs on the left side were broken. The patient received standard pain medication and was instructed to sleep in a recliner chair. At night the patient attempted to sleep in a recliner chair. With each breath there was a sharp pain in his side which prevented him from sleeping. He could feel his broken ribs moving during breathing. The patient then went to his bed to lie down and try to sleep, but experienced the same pain during breathing, and movement of the broken portion of his ribs, and was not able to sleep. The patient had previously, prior to his fall and resulting broken ribs, been prescribed a positive airway pressure device (BPAP) for the treatment of sleep apnea. Later in the night the patient attached his BPAP machine to prevent any sleep apnea in the event he was able to fall asleep. When the patient turned on the device, it completely relieved the pain associated with breathing as the patient adjusted the pressure to 16 cm of water. The patient then shut off the BPAP machine and the pain associated with breathing returned immediately. The patient then repeated this process six more times with the same results. The patient then left the machine on for the rest of the night and was able to sleep without any pain or discomfort related to his broken ribs. For the next five days the patient used the BPAP machine set at a positive airway pressure which prevented pain during breathing, and also used the BPAP machine at night while sleeping. After five days, the patient could no longer feel the broken portion of his ribs moving during normal breathing in the absence of the BPAP machine. Thereafter, he was able to resume his normal work activities, did not require the further use of the BPAP machine to prevent pain during breathing, and has had no such pain since.

The mechanism of action whereby continuous positive airway pressure relieves pain during breathing in patients with broken ribs is unknown. It may be that the continuous fixed positive airway pressure in the lungs created by the device produced an internal splinting of the chest wall resulting in pain relief and accelerated healing and knitting of the broken ribs. The continuous positive airway pressure in the lung may also produce alveolar recruitment thereby preventing the atelectasis and pneumonia that might result as a complication of fractured ribs. Any suitable type of positive pressure airway device known in the art may be used in the method of the present invention. The use of the positive pressure airway device may be needed for only one or two days to promote healing of the ribs sufficiently so that the device is no longer required for the duration of the healing period.

While the invention has been shown and described in some detail with reference to specific exemplary embodiments, there is no intention that the invention be limited to such detail. On the contrary, the invention is intended to include any alternative or equivalent embodiments that fall within the spirit and scope of the invention as described herein.

With respect to the above description then, it is to be realized that the optimum dimensional relationships for devices used in the method of the invention, to include variations in size, materials, shape, form, function and manner of operation, assembly and use, are deemed readily apparent and obvious to one skilled in the art, and all equivalent relationships to those described in the specification are intended to be encompassed by the present invention. Therefore, the foregoing is considered as illustrative only of the principles of the invention. Further, since numerous modifications and changes will readily occur to those skilled in the art, it is not desired to limit the invention to the exact method and operation described, and accordingly, all suitable modifications and equivalents may be resorted to, falling within the scope of the invention.

1. A method of relieving pain produced by breathing in a patient with fractured ribs, comprising the steps of:
1) providing a positive airway pressure machine;
2) attaching said positive airway pressure machine to the nose, mouth, or both of said patient;
3) turning said positive airway pressure on to produce a positive airway pressure in the airway or airways of the lungs of said patient; and
4) adjusting the positive airway pressure produced by said positive airway pressure machine to a level where the positive airway pressure relieves pain produce by breathing in said patient.

2. The method of claim 1 wherein the step of adjusting is performed by said patient.

3. The method of claim 1 further comprising using said method until no movement of the fractured portion of the ribs is detectable by said patient during normal breathing in the absence of said positive airway pressure machine.

4. The method of claim 1 wherein the method accelerates the healing of the fractured ribs of said patient.

5. A method of relieving pain produced by breathing in a patient with fractured ribs, comprising the steps of:
1) providing a positive airway pressure machine;
2) attaching said positive airway pressure machine to the nose, mouth, or both of said patient;
3) turning said positive airway pressure on to produce a positive airway pressure in the airway or airways of the lungs of said patient; and
4) adjusting by said patient the positive airway pressure produced by said positive airway pressure machine to a level where the positive airway pressure relieves pain produce by breathing in said patient.

6. The method of claim 5 further comprising using said method until no movement of the fractured portion of the ribs is detectable by said patient during normal breathing in the absence of said positive airway pressure machine.

7. The method of claim 5 wherein the method accelerates the healing of the fractured ribs of said patient.

8. A method of relieving pain produced by breathing in a patient with fractured ribs, comprising the steps of:
1) providing a non-ventilating positive airway pressure machine;
2) attaching said non-ventilating positive airway pressure machine to the nose, mouth, or both of said patient;
3) turning said non-ventilating positive airway pressure on to produce a positive airway pressure in the airway or airways of the lungs of said patient;
4) adjusting by said patient the positive airway pressure produced by said non-ventilating positive airway pressure machine to a level where the positive airway pressure relieves pain produce by breathing in said patient; and
5) using said method for at least two days or until no movement of the fractured portion of the ribs is detectable by said patient during normal breathing in the absence of said non-ventilating positive airway pressure machine.

9. The method of claim 8 wherein said method accelerates the healing of the fractured ribs of said patient.
